(19) United States
(12) Patent Application Publication
(10) Pub. No.: US 2021/0277412 A1
(43) Pub. Date: Sep. 9, 2021

(54) PLANT PRODUCED PORCINE CIRCOVIRUS PSEUDOVIRION

(71) Applicant: UNIVERSITY OF CAPE TOWN, Cape Town (ZA)

(72) Inventors: Edward Peter Rybicki, Cape Town (ZA); Guy Louis Regnard, Cape Town (ZA); Inga Isabel Hitzeroth, Cape Town (ZA)

(21) Appl. No.: 17/251,698

(22) PCT Filed: Jun. 11, 2019
(86) PCT No.: PCT/IB2019/054855

§ 371 Date: Dec. 11, 2020

(30) Foreign Application Priority Data
Jun. 11, 2018 (GB) 1809563.8

(51) Int. Cl. C12N 15/82; C12N 7/00; G01N 33/569; A61K 39/12

(52) U.S. Cl. CPC C12N 15/8258; C12N 7/00; G01N 33/56983; G01N 2333/01; C12N 2750/10051; C12N 2750/10034; C12N 2750/10043; A61K 39/12

(57) ABSTRACT

The present invention relates to methods of producing porcine circovirus (PCV) pseudovirions in plant cells, the plant-produced PCV pseudovirions, a neutralisation assay using the plant-produced PCV pseudovirions and pharmaceutical compositions comprising the plant produced PCV pseudovirions. In particular, the method of the invention relates to introducing expression vectors, replicating vectors and nucleic acids into the plant cell and allowing for expression of capsid proteins and replication of the replicating vector. The expressed PCV capsid polypeptides assemble, together with a single-stranded copy of the replicating vector and encapsidate it as a pseudogenome to produce a PCV pseudovirion.

### BACKGROUND OF THE INVENTION

The present invention relates to methods of producing porcine circovirus (PCV) pseudovirions in plant cells, the plant-produced PCV pseudovirions, a neutralisation assay using the plant-produced PCV pseudovirions and pharmaceutical compositions comprising the plant produced PCV pseudovirions.

Porcine circovirus type 2 (PCV-2) is the main causative agent associated with diseases collectively known as porcine circovirus associated disease (PCAD). There is a significant economic strain on the global swine industry due to PCAD, and to the high production costs of commercial PCV-2 vaccines. After the introduction of PCV-2 vaccines over a decade ago, effective control of new PCV-2 infections and its associated diseases has been achieved. Unfortunately, due to genotype prevalence shifts the currently available vaccines are proving less successful where vaccinated herds presenting with PCVAD often carry other PCV-2 genotypes. These prevalent PCV-2 genotypes are thus not effectively covered by currently available vaccines and the recent characterisation of a PCV-3 presents an additional threat. PCV-2 particles containing artificial genomes (pseudovirions, PsVs) may elicit stronger antibody and cellular immune responses and have the capacity to deliver exogenous genes into host cells that can express additional disease antigens. The successful construction of PsVs could allow for simultaneous coverage of different PCV-2 genotypes or other common swine virus pathogens, where if the DNA of other genotypes or viruses can successfully be packaged inside the particles, a single-use broad vaccine would be generated. Delivery vehicles must be efficient, easy to prepare and modify and immunogenic, so efforts should thus be ongoing to produce these next generation vaccines that could provide a wider range of cross-neutralising immunity.

Recent advances in the use of PsVs as vaccine delivery and antigen presenting agents are promising, but these are highly expensive to produce in mammalian cell lines, so affordable production systems need to be explored if PsV-based vaccines are going to be commercially viable. Plants have the flexibility to easily co-express different genes allowing for the construction of new bionanomaterials while using existing approaches based on plant virus-like particles (VLPs). A notable concern in the production of PsVs is the possible risk of non-selectively packaging potentially infective DNA present in mammalian cell lines; however, if PsVs are produced in plants the packaging of non-specific DNA from plant cells poses an insignificant risk for transmission of unsuspected pathogens or of oncogenes.

### SUMMARY OF THE INVENTION

The present invention relates to methods of producing porcine circovirus (PCV) pseudovirions in plant cells, the plant-produced PCV pseudovirions per se, a neutralisation assay using the plant-produced PCV pseudovirions and pharmaceutical compositions comprising the plant produced PCV pseudovirions.

According to a first aspect of the invention there is provided for a method for producing a porcine circovirus (PCV) pseudovirion in a plant cell, the method comprising the steps of (i) introducing into the plant cell (a) an expression vector comprising a first nucleic acid encoding a PCV capsid polypeptide; and (b) a replicating vector derived from a ssDNA virus comprising at least two origins of replication (Ori) sequences recognised by a viral replication regulatory protein, the replicating vector further comprising a second nucleic acid encoding a heterologous polypeptide, and (c) a third nucleic acid encoding a viral replication regulatory protein, wherein replication of the replicating vector is initiated by the viral replication regulatory protein, and wherein the second nucleic acid is operably linked to a regulatory sequence which allows for the expression of the heterologous polypeptide in a mammalian cell; (ii) expressing the PCV capsid polypeptide and the viral replication regulatory protein in the plant cell, and (iii) replicating the replicating vector from the Ori sequence recognised by the viral replication regulatory protein in the plant cell, in order to produce a high copy number of a pseudogenome comprising the second nucleic acid, wherein the expressed PCV capsid polypeptides assemble, together with a single-stranded copy of the pseudogenome and encapsidate the pseudogenome to produce a PCV pseudovirion.

In one embodiment of the invention the first nucleic acid is operably linked to regulatory sequences that allow for expression of the PCV capsid polypeptide in the plant cell.

In another embodiment of the invention the viral replication regulatory protein is expressed from at least one of the group selected from (i) a nucleic acid sequence contained on the replicating vector; (ii) a nucleic acid sequence contained on the at least one expression vector; (iii) a nucleic acid sequence contained on an independent vector, not being the vector of (i) or (ii) above; or (iv) a nucleic acid sequence integrated into the genomic DNA of the plant cell, wherein expression of the viral replication regulatory protein in the presence of the replicating vector results in replication of the replicating vector to produce a high copy number of the pseudogenome in the plant cell.

In a further embodiment of the invention the second nucleic acid encoding the heterologous polypeptide comprises a gene selected from the group consisting of a reporter gene, a therapeutic gene, a gene encoding an antigenic polypeptide, a gene encoding a hormone, an antibody or an enzyme. Preferably, the gene encoding the heterologous polypeptide is a reporter gene selected from a luciferase gene, a secreted alkaline phosphatase gene, a gene encoding a fluorescent protein or a horseradish peroxidase gene.

In yet a further embodiment of the invention the method further comprises a step of recovering the PCV pseudovirion from the plant cell.

According to a second aspect of the invention there is provided for an assay for detecting the presence of a neutralising antibody to PCV in a subject, the assay including the steps of: (i) combining a PCV pseudovirion produced according to the first aspect of the invention, with a biological sample from the subject to form a biological sample composition, wherein the heterologous polypeptide is a reporter polypeptide; and (ii) combining a PCV pseudovirion produced according to the method of the first aspect of the invention, with a control biological sample, wherein the control biological sample does not contain a PCV neutralising antibody, to form a control sample composition, wherein the heterologous polypeptide is a reporter polypeptide; (iii) contacting and incubating a mammalian cell capable of being infected with PCV with the biological sample composition of (i) or the control sample composition of (ii); and (iv) assaying the expression of the reporter polypeptide, wherein decreased expression of the reporter polypeptide in the mammalian cells contacted with the biological sample composition, as compared to mammalian cells contacted with the control sample composition is indicative of the presence of a PCV neutralising antibody in the biological sample.

In one embodiment of the invention the reporter polypeptide is selected from a protein expressed from a luciferase gene, a secreted alkaline phosphatase gene, a gene encoding a fluorescent protein or a horse radish peroxidase gene.

In a preferred embodiment of the invention the subject is a pig.

According to a third aspect of the invention there is provided for a PCV pseudovirion produced according to the method of the first aspect of the invention, the PCV pseudovirion comprising a capsid, wherein the capsid comprises the PCV capsid protein, wherein the capsid encapsidates the pseudogenome comprising the second nucleic acid encoding the heterologous polypeptide, wherein the second nucleic acid is operably linked to a regulatory sequence that allows for the expression of the heterologous polypeptide in a mammalian cell, wherein replication of the replicating vector is initiated from the Ori sequence recognised by the viral replication regulatory protein, and wherein the PCV pseudovirion is produced in and recovered from the plant cell.

In one embodiment of the third aspect of the invention, replication of the pseudogenome may be initiated in a mammalian cell infected by the PCV pseudovirion in the presence of a viral replication regulatory protein, wherein the viral replication regulatory protein is a replication associated (Rep) protein.

In a further embodiment of the invention the viral replication regulatory protein is encoded by a nucleic acid sequence operably linked to a regulatory sequence that allows for the expression of the regulatory protein in the mammalian cell, wherein the viral replication regulatory protein may be expressed from any one of the group consisting of: (i) a nucleic acid sequence contained on the pseudogenome; (ii) a nucleic acid sequence contained on an independent vector; or (iii) a nucleic acid sequence integrated into the genomic DNA of the mammalian cell, wherein expression of the viral replication regulatory protein in the mammalian cell results in the replication of the pseudogenome.

In a preferred embodiment of the invention the heterologous polypeptide is expressed from a gene selected from the group consisting of a reporter gene, a therapeutic gene, a gene encoding an antigenic polypeptide, a gene encoding a hormone, an antibody or an enzyme.

In a fourth aspect of the invention there is provided for a pharmaceutical composition comprising a PCV pseudovirion produced by the method of the first aspect of the invention or the PCV pseudovirion of the third aspect of the invention and a pharmaceutically acceptable carrier or adjuvant.

### BRIEF DESCRIPTION OF THE FIGURES

Non-limiting embodiments of the invention will now be described by way of example only and with reference to the following figures:

FIG. 1: Schematic representation of the difference between producing a human papillomavirus capsid protein packaging of recombinant DNA (Top) and producing a porcine circovirus capsid protein packaging of recombinant DNA (Bottom). The PCV capsid protein will only encapsidate ssDNA and by binding to the ssDNA it prevents conversion of ssDNA to dsDNA

FIG. 2: Representation of PCV-2 genome-like plant expression vector (PCVGp) and amplicon. A—Replication module capable of releasing a circular ssDNA amplicon similar in size to the PCV-2 genome with PCV-2 genome-like amplicon expression cassette shown, LIR: BeYDV long intergenic region, SV40 enh/pA: simian virus 40 enhancer and poly-adenylation site, YFP: yellow fluorescence protein gene, SIR: BeYDV short intergenic region, BeYDV rep/repA: BeYDV replication associated protein gene, CaMV enh/pA: cauliflower mosaic virus promoter and polyadenylation site. B—pEAQ-HT derived expression cassette with dark grey arrows representing areas essential for plasmid growth and replication OriV: pRK2 replication origin, ColE1: pBR322 replication origin, NPT: neomycin phosphotransferase, TrfA: replication essential locus, LB and RB: left and right borders (Sainsbury et al. 2009). C—PCV-2 genome-like amplicon following DNA release and circularisation in plants.

FIG. 3: Confirmation of PCV-2 genome-like amplicon accumulation in plants. A—Electrophoresis gel of restriction enzyme digested RCA product from total DNA extracted from plant tissue infiltrated with the PCVGp vector. The bands indicate the size of the expected PCV-2 amplicon at 1803 bp over seven days (arrow) with the full 9763 bp PCVGp, no template (−) control and DNA ladder in nucleotide base pairs. B—Gene copy number from plant tissue infiltrated with PCVGp over time. The bars indicate two independent experiments of total YFP gene copy number over seven days after an A. tumefaciens infiltration.

FIG. 4: Confirmation of YFP functionality in HEK-293TT and PK-15 cells. Images represent HEK-293TT and PK-15 cells at 40× magnification, 48 h after FuGene® transfection with either the full PCVGp or the plant-made, rolling circle amplified PCV-2 amplicon cut with Pstl and re-ligated. Images were viewed under bright field or YFP filter block on a Nikon Ti-E inverted microscope with auto exposure.

FIG. 5: Purification and identification of plant produced PCV-2 pseudovirions. A—Coomassie blue stained SDS-PAGE gel with corresponding immunoblot detected using anti-PCV-2 CP rabbit serum of co-infiltrated plant samples. The collected sucrose gradient fractions 1-4 and resuspended pellet (P) after ultracentrifuge spin, with PCVGp only negative control (−). The marker is in kilodaltons. B—Transmission electron microscopy images of plant produced PCV-2 VLPs alone or with PCVGp. The pellet which formed after sucrose gradient purification was resuspended in PBS, loaded onto carbon coated copper grids and stained with uranyl acetate for TEM. The scale bar is 50 nm. C—Electrophoresis gel of plant expressed samples treated with a nuclease, protease, both or neither. The samples underwent RCA after treatment, were digested with Pstl and resolved on an electrophoresis gel to determine the presence of ˜1800 bp PCV-2 amplicon and its packaging in PCV-2 VLPs. The DNA ladder is in nucleotide base pairs.

FIG. 6: Assessment of PCV-2 genome-like amplicon entry in mammalian cells. Images represent 10× magnified HEK-293TT cells 48 h after exposure to the PCV-2 VLP alone, plant-made PCV-2 amplicon alone or together without transfection reagent viewed under bright field or YFP filter block on a Nikon Ti-E inverted microscope with auto exposure.

FIG. 7: Amplicons containing BeYDV SIR and LIR, a simian virus 40 (SV40) cassette (AF362548) for the expression in mammalian cells of the enhanced yellow-green variant of the Aequorea victoria fluorescent protein (YFP) gene from the pEYFP-c1 vector (catalog number: 6005-1) Clontech Laboratories (California, USA). This construct, excluding the YFP gene and BeYDV rep/repA gene was synthesised by GenScript® (SEQ ID NO:1).

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

PCV-2 particles containing artificial genomes elicit strong antibody and cellular immune responses and have the added capacity to deliver exogenous genes into host cells that can express additional disease antigens. The present inventors have shown that it is possible to express the PCV-2 capsid protein (CP) and to produce circular ssDNA PCV-2-genome-like amplicons (PCVGp) in Nicotiana benthamiana plants so that co-expression of the CP and PCVGp would produce pseudovirions.

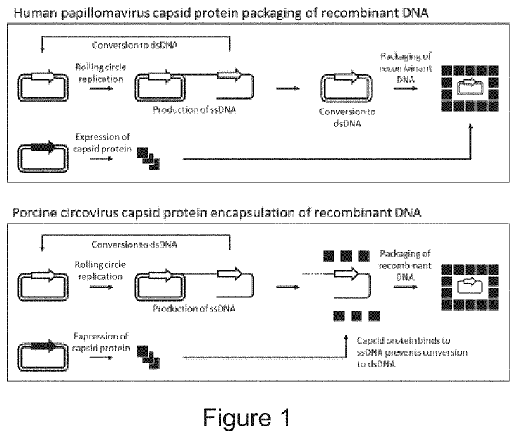

The present invention differs from pseudovirions produced in the art, and specifically from the production of papillomavirus pseudovirions in plants, in that it depends upon a specific interaction of the single co-expressed circovirus coat protein with a nascent single-stranded DNA resulting from rolling circle replication of a circovirus-derived amplicon in plant cells, to form particles containing single-stranded rather than double-stranded DNA. In the case of HPV or other PVs, the two co-expressed coat proteins assemble with any circular double-stranded DNA in the correct size range to form pseudovirions (FIG. 1). In this case, although a circular double-stranded DNA replicative form amplicon (pseudogenome) is made in plants similarly to the PV pseudovirion production method, the circovirus coat protein cannot encapsidate this form and instead sequesters the transient ssDNA form to form pseudovirions.

Provided herein is a method for producing a porcine circovirus (PCV) pseudovirion in a plant cell. “Circoviruses” are single-stranded DNA viruses from the family Circoviridae. Birds and pigs serve as natural hosts, though dogs have been shown to be infected as well. A “VLP” or “virus-like particle” refers to the capsid-like structure which results from the assembly of the PCV capsid protein. These structures are antigenically and morphologically similar to actual PCV virus particles or virions. Virus-like particles do not include viral genetic material; accordingly, these particles are not infectious.

The term “pseudovirion” or “PsV” refers to a circovirus virus-like particle including the circovirus capsid protein in which a circovirus-derived single stranded DNA plasmid or vector containing a heterologous gene of interest has been encapsidated. The pseudovirions of the invention contain non-native genetic material which can be transferred by the virus to an animal cell, preferably a mammalian cell, and most preferably to a porcine cell. The non-native genetic material may include a plasmid encoding a reporter gene, a therapeutic gene, a gene encoding an antigenic polypeptide, a gene encoding a hormone, an antibody or an enzyme and/or any other heterologous gene of interest under the control of a mammalian promoter, which can be delivered to a mammalian cell by the pseudovirion. In this specification “encapsidated” refers to the plasmid or vector being enclosed within the capsid of the virus-like particle.

The term “protein” should be read to include “peptide” and “polypeptide” and vice versa.

The method of the invention includes the steps of introducing a first polynucleotide encoding a PCV polypeptide into a plant cell. Preferably, the first polynucleotide is contained on a vector.

The term “vector” refers to some means by which polynucleotides or gene sequences can be introduced into a cell. There are various types of vectors known in the art including plasmids, viruses, bacteriophages and cosmids. Generally polynucleotides or gene sequences are introduced into a vector by means of a cassette. The term “cassette” refers to a polynucleotide or gene sequence that is expressed from a vector, for example, the polynucleotide or gene sequences encoding the PCV capsid protein. A cassette generally comprises a gene sequence inserted into a vector, which in some embodiments, provides regulatory sequences for expressing the polynucleotide or gene sequences. In other embodiments, the polynucleotide or gene sequence provides the regulatory sequences for its expression. In further embodiments, the vector provides some regulatory sequences and the nucleotide or gene sequence provides other regulatory sequences. “Regulatory sequences” include but are not limited to promoters, transcription termination sequences, enhancers, splice acceptors, donor sequences, introns, ribosome binding sequences, poly(A) addition sequences, and/or origins of replication.

The method further includes the step of introducing a second polynucleotide sequence into a plant cell. The second polynucleotide sequence is in the form of an amplicon sequence which is contained within a larger vector. The amplicon itself contains a polynucleotide encoding a heterologous polypeptide of interest and a mammalian promoter or a promoter which is capable of promoting expression of a protein in a mammalian cell. In order for amplification of the amplicon encoding the heterologous polypeptide of interest to proceed, replicational release of the amplicon from the larger vector must occur in a plant cell. This is achieved by action of a plant ssDNA geminivirus-derived Rep (replication associated) protein, which nicks the amplicon sequence in one strand at one of two characteristic nonanucleotide motifs included in the vector, binds covalently to the newly exposed 5′-terminus, and enables host repair or other DNA polymerases to extend the strand from the new 3′-terminus, simultaneously displacing the nicked or amplicon strand as a single-stranded DNA. Once this is released from the larger vector by Rep cleavage at the second nonanucleotide sequence, the amplicon ssDNA circularises due to self-complementarity around the nonanucleotide sequence, and is ligated to form a circular ssDNA form by action of the attached Rep. The amplicon, together with an expression cassette comprising a polynucleotide or gene sequence encoding the heterologous polypeptide of interest and associated promoter, is rendered double-stranded by host repair polymerases, and further replicated by action of Rep to high copy number in the plant cell.

The expression of the PCV capsid polypeptide in the plant cell results in the polypeptide self-assembling into a virus-like particles in the cell. As a result of the high copy number of the amplicon in the cell copies of the ssDNA version of the amplicon are specifically encapsidated directly into the virus-like particle during assembly in the cell to form pseudovirions. This is in contrast to indirect methods of incorporating a polynucleotide of interest into a pseudovirion by chemically or mechanically separating the virus-like particles and introducing a polynucleotide of interest into the virus-like particle to form a pseudovirion, and does not allow incorporation of dsDNA forms.

The replicating vector is incapable of replicating in a mammalian cell: this is due to the fact that while the plant virus-derived Rep/RepA protein may be capable of initiating replication of the amplicon in an animal cell, preferably a mammalian cell, in the vector system described the Rep/RepA protein is expressed from a plant-specific promoter sequence which cannot be recognised in a mammalian cell. The Rep/RepA gene is also not present on the pseudogenome that is created. On the other hand, the gene encoding the heterologous polypeptide is, however, capable of being expressed in a mammalian cell because it is under the control of a well-characterised mammalian promoter as part of the expression cassette.

As used herein the term “viral replication regulatory protein” refers to a protein which is capable of initiating replication of the replication vector at its origin of replication (Ori), in a preferred embodiment the viral replication regulatory protein is Rep/RepA, however it will be appreciated by those of skill in the art that any viral replication regulatory protein which is capable of initiating replication of the replicating vector may be used.

It will be appreciated by a person skilled in the art that the gene encoding the Rep/RepA protein may be contained on the replicating vector, on another vector (such as a vector containing a cassette encoding the PCV capsid polypeptide), it may further be integrated into the DNA of the plant cell in which the pseudovirions are produced, or it may be integrated, together with a suitable promoter, into the DNA of a mammalian cell into which the carrier vector is introduced by the pseudovirion. The presence of the Rep/Rep A protein in the plant cell may result in the initiation of replication of the replicating vector and production of the amplicon to high copy number. Alternatively the presence of the Rep/RepA protein in a mammalian cell may result in the production of the amplicon to high copy number in the target cell.

The gene encoding the heterologous polypeptide contained on the amplicon may include a gene selected from the group consisting of a reporter gene, a therapeutic gene and possibly other genes related to desirable human or animal vaccine proteins, including hormone proteins or hormone peptides, antibodies or enzymes.

It will be appreciated that a “reporter gene” may be selected from any nucleic acid encoding a polypeptide or protein whose transcription, translation and/or post-translation activity can be detected. Examples of reporter genes include, but are not limited to, genes for luciferase, secreted alkaline phosphatase, green fluorescent protein, beta-galactosidase, and the like. The expression of the reporter polypeptide is used in the present invention as an indicator of the presence of neutralising antibodies to PCV in a sample. The pseudovirions of the invention can thus be used in a neutralisation assay for detection of neutralising antibodies to PCV in a subject.

It will also be appreciated that the amplicon may be derived from any single-stranded DNA virus of plants, including, but not limited to, geminiviruses and nanoviruses, as well as from bird and mammalian circoviruses, or from parvoviruses, and/or bacterial ssDNA viruses or bacterial plasmids that replicate via a similar rolling circle DNA replication strategy. All that is required is a viral replication regulatory protein, such as Rep/RepA or an equivalent, expressed in the presence of a DNA construct carried in a larger plasmid, which incorporates at least one origin of replication sequence (Ori) recognised by the viral replication regulatory protein so as to allow the initiation of rolling circle replication.

The pseudovirion neutralisation assay of the invention could be used for the development of a PCV pseudovirus neutralisation kit which could be used to test the effectiveness of potential PCV vaccine candidates and determine prior exposure of animals to PCV.

The delivery of the amplicon from the pseudovirion to a mammalian cell is a clear indicator that the pseudovirions of the invention are capable of being used as DNA delivery vehicles for the purposes of gene therapy.

Production of the pseudovirions of the invention in plants has certain benefits over the current mammalian cell production methods. Among others the cost of production of plant derived pseudovirions is substantially lower than the cost of production in mammalian cells. Currently, pseudovirions are only produced in mammalian cancer-derived cultured cells: this production method poses certain safety issues in that the pseudovirions could encapsidate oncogenes from the cell lines. This could result in a subject who is treated with these pseudovirions being “infected” with cancer-causing genes. Further, propagation of pseudovirions in mammalian cell lines could result in other viruses and/or contaminants being encapsidated in the capsid.

The method of production of the pseudovirions of the invention in plants is a simple process and removes the possibility of oncogene or mammalian virus contamination. The process is also highly scalable. Further, should plant virus-derived replicating DNA be encapsidated into the pseudovirions of the invention this plant virus-derived DNA will not be capable of replicating in mammalian cells or of combining with other mammalian viruses or transposon like sequences.

The following examples are offered by way of illustration and not by way of limitation.

### Example 1

PCV-2 Genome-Like Plant Expression Vector Construction

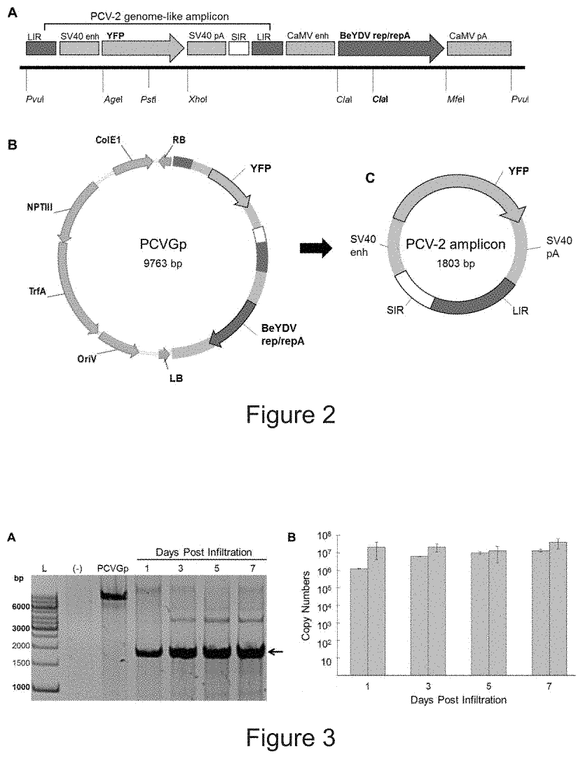

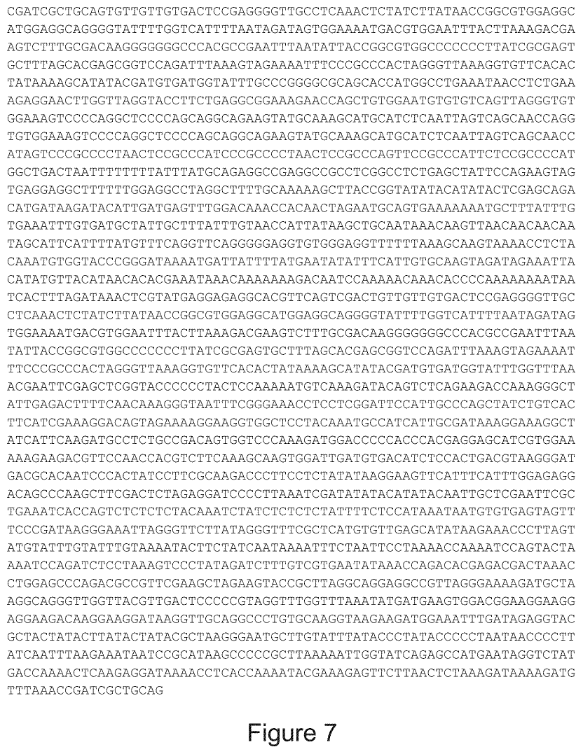

A plant vector module capable of releasing a circular ssDNA amplicon similar in size to the PCV-2 genome (1800 nucleotides) was designed by Dr Guy Regnard in the Biopharming Research Unit (FIG. 2). The pseudogenome was specifically designed to be as small as possible in order to ensure encapsidation. The vector consisted of replication elements from BeYDV capable of releasing circular amplicons through rolling circle replication in plants, but which are incapable of replication in mammalian cells. These replication elements included a repeated BeYDV (GenBank accession number DQ458791) LIR, BeYDV short intergenic region (SIR) and the BeYDV rep/repA. The rep/repA was placed under the control of the CaMV 35S expression cassette from the pEAQ-HT vector (Sainsbury et al. 2009); upon translation Rep/RepA binds to the LIR to initiate rolling circle replication, thereby releasing amplicons. The amplicons contain BeYDV SIR and LIR, a simian virus 40 (SV40) cassette (AF362548) for the expression in mammalian cells of the enhanced yellow-green variant of the Aequorea victoria fluorescent protein (YFP) gene from the pEYFP-c1 vector (catalog number: 6005-1) Clontech Laboratories (California, USA). This construct, excluding the YFP gene and BeYDV rep/repA gene was synthesised by GenScript® (SEQ ID NO:1, FIG. 7) provided on the pUC57 vector cloned into PvuI sites.

The first step was to clone the YFP into the vector using Agel and Xhol restriction enzymes (FIG. 2). After isolation on a 1% TBE agarose gel based on size, gel extractions, ligation and E. coli transformations followed. Colonies containing the YFP gene were screened by colony PCR using a LIR-specific forward primer 5′-ACG ATG TGA TGG TAT TTG CCC G-3′ (SEQ ID NO:2) and a SIR-specific reverse primer 5′-CGT GCC TCT CCT CAT ACG AG-3′ (SEQ ID NO:3). The next step was to clone the BeYDV rep/repA into the vector now containing the YFP gene. This had to be done by partial digestion using 0.17 units of C/al and 1.00 units of Mfel as the BeYDV rep/repA sequence contained a C/al site (FIG. 2). The vector with YFP gene incorporated was also digested with C/al and Mfel. Based on size, the resulting DNA fragments were isolated, ligated and E. coli transformed. To screen for BeYDV rep/repA positive clones a BeYDV rep/repA specific forward primer 5′-GAA ATC GAT ATG CCT TCT GCT AGT AAG A-3′ (SEQ ID NO:4) and reverse primer 5′-CTT CAA TTG TCA GTG ACT CGA CGA TTC-3′ (SEQ ID NO:5) were used for colony PCR.

Finally, this entire replicating module was cloned into the pEAQ-HT expression vector. PvuI sites were specifically added to the 5′ and 3′ end of module to allow replacement of pEAQ-HT expression cassette (Sainsbury et al. 2009) between the left and right borders (FIG. 2). pEAQ-HT was linearized with PvuI, and the replicating module was excised from the pUC57 vetor by PvuI digestion. DNA fragments were isolated on a 1% agarose gel based on size, purified, and pEAQ-HT vector was dephosphorylated. This was followed by ligation and E. coli transformations. This gave rise to the novel PCV-2 genome-like plant expression vector (PCVGp). The LIR/SIR primers and BeYDV rep/repA primers were used for colony PCR confirmations. All constructs were verified by sequencing (Macrogen).

A. tumefaciens Transformations, Plant Infiltrations and Protein Purification

The Agrobacterium tumefaciens LBA4404 strain was electroporated for transformation with DNA constructs. The transformants were screened by PCR using primer sets for the LIR/SIR and BeYDV rep/repA to select positive recombinant clones. These were inoculated into LB broth containing 30 μg/mL kanamycin and 50 μg/mL rifampicin antibiotics. The cultures were supplemented with 2 mM MgSO4 to prevent cell clumping.

N. benthamiana plant leaves were vacuum infiltrated for medium scale expression studies with the PCV-2 CP gene in the pEAQ-HT vector at an Agrobacterium OD₆₀₀ of 1.0 and PCVGp at an OD₆₀₀ of 0.5 together as co-infiltrations for pseudovirion formation studies. The plant lysate was clarified and the supernatant containing the PCV-2 CP was filtered through 4 layers of Miracloth® (Merck, USA). A sucrose gradient was prepared by underlaying dilutions of 6 mL (45%) and 2 mL (65%) in Thinwall 38 mL Ulta-Clear™ ultracentrifuge tubes (Beckman). The clarified plant supernatant was loaded onto the gradient, and centrifuged using a Beckman SW 32 Ti rotor at 120 000×g and 4° C. for 4 h. Two millilitre fractions were collected in microcentrifuge tubes and the pellet was resuspended in 1 mL PBS. A sample from each fraction and the pellet was boiled for 5 minutes with sample application buffer and analysed with SDS-PAGE followed by Coomassie brilliant blue staining or immunoblotting.

Nuclease and Protease Treatments

Purified pellet samples resuspended in 100 μL PBS were treated with 50 units of Benzonase® Nuclease (Sigma-Aldrich®) and incubated at 37° C. for 10 minutes, this was followed by 60 units of Proteinase K (Sigma-Aldrich®) treatment and 55° C. incubation for 3 h. All the samples underwent the same temperature incubations regardless of whether they were treated with either or both or neither of nuclease and proteinase and a PCR clean-up (Qiagen, Germany) as per manufacturers protocol was done before the RCA studies.

Rolling Circle Amplification

Total DNA was isolated from plant tissue using the Extract-N-Amp™ Plant PCR Kits (Sigma-Aldrich®) as per manufactures instructions. Briefly, one leaf disc was cut using the cap of a standard 1.5 mL microcentrifuge tube, resuspended in 100 μl extraction buffer and heated for 10 minutes at 95° C. After heating, 100 μL of dilution buffer was added, the leaf disc was discarded and the extracted total plant DNA mixture was stored at 4° C. until use.

Rolling circle amplifications were performed on plant DNA extracts with the PCVGp vector at 1, 3, 5 and 7 days post infiltration using an illustra TempliPhi 100 Amplification Kit (Amersham Biosciences, UK). This method uses bacteriophage ϕ29 DNA polymerase and random hexamers that bind non-specifically to exponentially amplify circular ss- or ds DNA templates by rolling circle amplification. Briefly, 1 μL of isolated total plant DNA was added to 5 μL of sample buffer and heated at 95° C. for 3 minutes. After cooling, 5 μL of reaction buffer as well as 0.2 μL of enzyme mix was added and the mixture was incubated at 30° C. for 18 h. After incubation, the polymerase is inactivated at 65° C. for 10 minutes and the samples stored at 4° C. or further analysed. Samples were digested with Pstl and resolved on an electrophoresis gel, the bands corresponding to the PCV-2 amplicon size 1803 bp were gel extracted and re-ligated for mammalian cell transfection studies or cloned into the pGEM®-3Zf(+) vector (Promega, Wis., USA) vector for sequencing (Macrogen).

Quantitative PCR

The Rotor-Gene RG-3000A (Qiagen) was used for real-time quantification PCR (qPCR) with a minor groove DNA binding LuminoCt® SYBR® Green qPCR ReadyMix™ (Sigma-Aldrich®) fluorescent dye. One microliter of undiluted total plant DNA was used as template in a 20 μL reaction mixture with a YFP specific forward primer 5′-CCC GAC AAC CAC TAC CTG AG-3′ (SEQ ID NO:6) and reverse primer 5′-GTC CAT GCC GAG AGT GAT CC-3′ (SEQ ID NO:7 (at 0.4 μM concentrations designed using the NCBI Primer-BLAST tool. The primers amplified a 117 bp YFP gene product. The PCVGp prepared from E. coli cells was used as control to construct the standard curve for analysis using Rotor-Gene™ 6000 software, version 1.7, build 87 (Qiagen). Quantification PCRs were always done in triplicate for each biological replicate with an initial 10 minute denaturation at 95° C. followed by 35 cycles of 15 s at 95° C., 15 s at 57° C. and 15 s at 72° C.

Mammalian Cell Culture Maintenance and Transfections

Human embryonic kidney 293 cell line (HEK-293TT) (ATCC® CRL-3216™, Virginia, USA) was sustained in Dulbecco's Modified Eagle's Medium (Invitrogen™) supplemented with 10% foetal calf serum, 2 mM L-glutamine, 100 IU/mL penicillin and 100 IU/mL streptomycin. A porcine kidney 15 cell line (PK-15) (ATCC® CCL-33™) was sustained in the same culture medium, but without L-glutamine. Cell passages were done bi-weekly with trypsin. The growth medium was removed from the cells and washed with phosphate buffered saline (PBS; 2.7 mM KCl, 10 mM Na₂HPO₄, 137 mM NaCl, pH 7.4) before trypsin-EDTA (0.05% trypsin and EDTA in PBS, pH 7.4) was added to each flask and left for 30 s (HEK-293TT) and 10 minutes (PK-15) to detach cells. Growth medium was used to neutralise the trypsin before the cell suspension was centrifuged for 2 minutes at 600×g and the cell pellet resuspended in adequate amount of growth medium. The cells were then passaged in a 1:10 dilution or seeded at a 1×10⁵ cells/mL density in 6 well plates (Sigma-Aldrich®) for experiments.

Transfection medium was prepared where 6 μL of FuGENE® HD (Promega) was used for every 2 μg of DNA in 100 μL of Dulbecco's Modified Eagle's Medium without supplements. Samples were mixed by vortex and incubated for 10 minutes at room temperature before adding total volume drop-wise to each well of ˜70% confluent cells. For the infection efficacy studies, a total of 100 μL plant-made and purified PCV-2 PsVs in PBS only were added per well and incubated for 48 h. The flasks and dishes were kept in an incubator at 37° C. with a humidified 5% CO₂ atmosphere.

Cell plates were visualised with the Nikon Ti-E Inverted Microscope (Japan) using the bright field lens, YFP filter block (lot number: C-162065). The YFP filter block has an excitation wavelength between 490-510 nm and emission wavelength of 520-550 nm. All images were taken with auto exposure at 10 or 40× magnification and from 24 h until 72 h after transfection.

SDS-PAGE, Immunoblotting and Transmission Electron Microscopy

SDS-PAGE and immunoblotting were done with 1:1000 dilution of the rabbit anti-PCV CP serum to probe the PCV-2 CP followed by a 1:5000 anti-rabbit secondary antibody conjugated to alkaline phosphatase (Sigma-Aldrich®). Glow discharged carbon-coated copper grids were prepared with samples of the sucrose density purified pellets resuspended in PBS. The grids were viewed at 27 000-50 000 magnification with a FEI Tecnai F20 transmission electron microscope at an accelerating voltage of 120 kV.

PCV-2 Amplicon Formation and DNA Replication Studies in Plants

N. benthamiana plants were infiltrated with PCVGp in LBA4404 A. tumefaciens and total DNA was extracted from the plant leaves after 1, 3, 5 and 7 days of infiltration. The presence and formation of a PCV-2 genome-like amplicon was confirmed using RCA from 1 dpi (FIG. 3). After 3 dpi, and up to 7 dpi there was a relatively stronger band at the expected size of 1803 bp compared to 1 dpi. The appearance of a second band at ˜3600 bp resulting from circular DNA also containing a Pstl restriction site was noted from 3 dpi onwards (FIG. 3). The difference in DNA levels was measured using qPCR, and confirmed a plateau in copy number at 10⁶ of the PCV-2 genome-like amplicon from 1 dpi, with a slight increase to 10⁷ copies after 3 dpi at which it remained until 7 dpi. Results presented are from two independent experiments (FIG. 3). The RCA band corresponding to the PCV-2 amplicon at 1803 bp was excised, gel purified and sent for sequencing: this confirmed the presence of the YFP gene, SV40 expression cassette, SIR and LIR in the correct order on the plant-made PCV-2 genome-like amplicon, and reporter gene functionality studies followed.

Reporter Gene Fluorescence Functionality in Mammalian Cell Lines

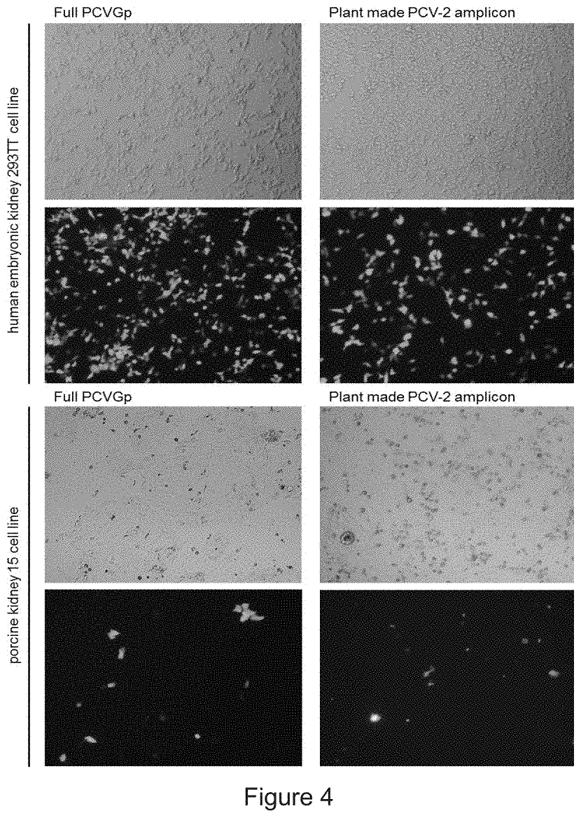

Mammalian HEK-293 and PK-15 cells were transfected using FuGene® with the PCVGp as well as the PCV-2 amplicon to test for the functionality of the SV40 cassette and YFP expression. YFP expression was detected for both the complete PCVGp and the plant made PCV-2 amplicon, with no visible reduction in cell growth. The transfection efficiency in the PK-15 cell line was markedly lower compared to the HEK-293 cell line (FIG. 4). After the confirmation of PCV-2 amplicon formation in plants and reporter gene functionality in mammalian cells, Agrobacterium co-infiltration with PCV-2 cp in pEAQ-HT vector and PCVGp was done to assess PsV formation in plants.

Pseudovirion Confirmation Studies

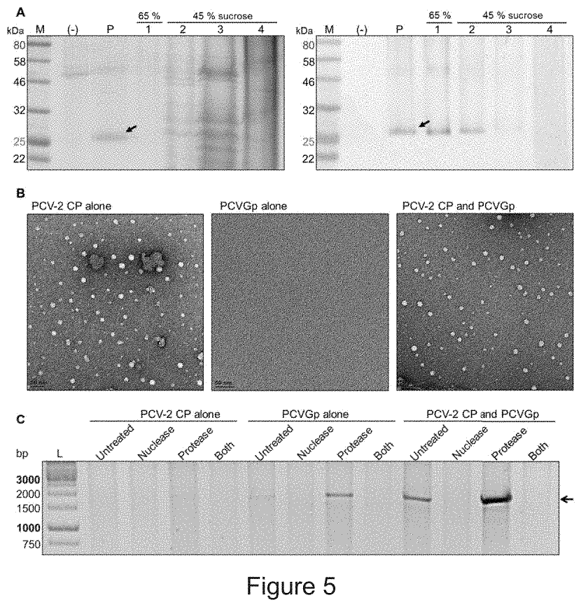

Plant leaves were single or co-infiltrated with the PCV-2 cp in pEAQ-HT and PCVGp, harvested at 4 dpi and sucrose density gradient centrifugation purifications were done. The pellet from the co-infiltrations showed a clear protein band at 27 kDa (FIG. 5A, lane P), with some protein also remaining in the 65% and 45% sucrose fractions (FIG. 5A, lane 1 and 2). Most of the plant proteins were captured in the 45% sucrose gradient (FIG. 5A, lane 2-4). The PCVGp only control was infiltrated, purified and pellets resuspended as for experimental samples, but no protein signal was seen.

The resuspended pellets were analysed using TEM and showed the probable formation of plant-produced PCV-2 VLPs. The VLP diameters ranged from 8 nm to 20 nm (analysis not shown), but average particle distribution from the captured images was −14 nm (FIG. 5B). The PCVGp only control did not show any particles. The protein samples were treated with a nuclease, protease or both and followed by RCA and restriction enzyme digestion studies to determine the presence and packaging of the 1803 bp PCV-2 amplicon inside the PCV-2 VLPs. The PCV-2 CP only samples did not have any amplicon signal after any combination of treatments (FIG. 5C). A DNA fragment was detected for the PCVGp only samples with and without protease treatment. The sample treated with protease produced a more intense DNA band. The samples obtained from the co-infiltrated plants with both the PCVGp and the PCV-2 CP in pEAQ-HT had signal after no treatment and robust signal at the expected amplicon size of ˜1800 bp after protease treatment (FIG. 5C). No signal was noted in any samples after it had undergone nuclease treatment.

Reporter Gene Delivery in Mammalian Cells

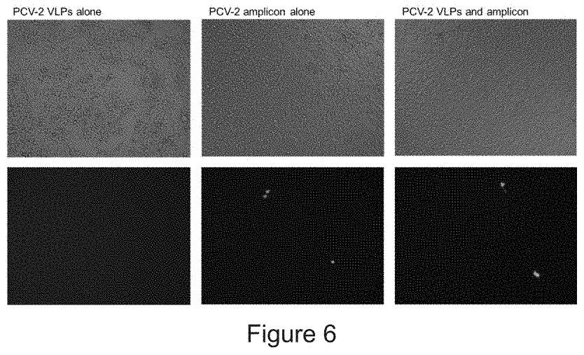

The infectivity of the presumptive plant-produced PCV-2 PsVs was determined with samples directly loaded onto HEK-293TT and PK-15 cells. Cells that were treated with the PCV-2 VLPs alone did not show any yellow fluorescence (FIG. 6). The cells that were treated with the plant-produced PCV-2 amplicons alone did show limited fluorescence 48 h after incubation, indicating the entry of these amplicons into cells without the PCV-2 VLP. The PCV-2 VLP with amplicons (PsVs) showed fluorescence at the same frequency as the PCV-2 amplicons alone (FIG. 6). The same studies were done on PK-15 cells but no fluorescence was detected up to 72-h after exposure (data not shown).

### Discussion

The present inventors have designed a novel geminivirus-based vector that can replicate in plants and be used to produce smaller plasmid-like amplicons in N. benthamiana to illustrate the potential for producing a PCV-2 DNA-based vaccine (FIG. 2). This vector uses rolling-circle replication mediated by a geminivirus Rep protein to amplify the gene-of-interest copy number in plants (Regnard et al. 2010). The PCV-2 dsDNA amplicons would serve as a source for a ssDNA pseudogenome to be packaged by PCV-2 CP to form PsVs in plants.

The formation of plant produced amplicons at the expected size of 1803 bp was confirmed with RCA (FIG. 3). The formation of circular DNA at approximately twice the size of the expected amplicon ˜3600 bp was also detected from RCA studies 3 dpi. This could be a dimeric form of the amplicon because of re-ligation and incomplete restriction enzyme digestion before the DNA was resolved. Alternatively, it could be due to recombination of DNA at the complementary stem loops in the LIR during amplification in the plant cells.

The YFP gene was cloned into the mammalian expression cassette of the PCV-2 amplicon to serve as a reporter gene for DNA delivery and expression studies. The YFP gene has the same nucleotide sequence length as the PCV-2 ORF2 to allow interchangeable gene sequence possibilities with minimum sequence length variation. The functionality of the expression cassette was confirmed by fluorescence in HEK-293TT and PK-15 cells transfected with the full PCVGp as well as plant-produced PCV-2 genome-like amplicons (FIG. 4). However, transfection in the PK-15 cell line was markedly lower compared to HEK-293TT cells, possibly as a result of DNA not being able to penetrate the nuclear envelop of the PK-15 cells by itself (Ren et al. 2016). The choice of SV40 expression cassette might further explain the marked difference in lower expression in the PK-15 cell-line. As the SV40 origin of replication is present on the SV40 expression cassette flanking the YFP gene here, much more expression and fluorescence was seen due to the presence of T antigen in the HEK-293TT cell line (Qin et al. 2010; Li et al. 2014). Previous PCV-2 DNA-based vaccine studies using the cytomegalovirus expression cassette and alternate commercial transfection reagents, have shown plasmid cell entry and expression in the PK-15 cell line (Sylla et al. 2014). These aspects are under consideration for future and ongoing studies.

The inventors co-infiltrated plants with the PCV-2 CP gene in the pEAQ-HT vector and PCVGp so that these would co-express PCV-2 VLPs with PCV-2 amplicons, and potentially package these to form PCV-2 PsVs. Electron microscopy confirmed the formation of plant-produced VLPs in plants where CP was co-expressed with PCV-2 amplicons (FIGS. 5B and 5C). After co-infiltration, the expression of PCV-2 CP and its formation into VLPs was confirmed but the packaging of PCV-2 amplicons needed to be determined as DNA is sometimes associated with the viral protein, but not encapsidated (Rossi et al. 2000). Samples were treated with nuclease to remove DNA associated with the virus particles on the outside but which had not been packaged. No amplicon was seen present in these samples compared to untreated samples, suggesting that the amplicons could be associated with VLPs, but not via packaging (FIG. 5C). Treatment with only protease revealed a marked increase in presence of amplicons which implies amplicon packaging, or that the digestion of proteins exposed more DNA for amplification.

A functioning PsV must be able to enter and successfully deliver its cargo to targeted cells with high transfection efficiency. To further determine if PsVs were forming, the infectivity of PCV-2 VLPs and delivery of the PCV-2 amplicon that would result in fluorescence, were tested on HEK-293TT and PK-15 cells. Fluorescence was detected in the HEK-293TT cell line.

In conclusion, the present inventors successfully developed a novel BeYDV-based replicating vector producing PCV-2 genome-like amplicons in plants that were encapsidated by PCV-2 coat protein to form pseudovirions, which were successfully used to express a YFP reporter gene in HEK-293TT and PK-15 cell lines.

### REFERENCES

Li, J. et al., 2014. Optimal transfection methods and comparison of PK-15 and Dulac cells for rescue of chimeric porcine circovirus type 1-2. Journal of virological methods, 208, pp. 90-95.
Qin, J. Y. et al., 2010. Systematic comparison of constitutive promoters and the doxycycline-inducible promoter. PloS one, 5(5), p.e10611.
Regnard, G. L. et al., 2010. High level protein expression in plants through the use of a novel autonomously replicating geminivirus shuttle vector. Plant biotechnology journal, 8(1), pp. 38-46.
Ren, L., Chen, X. & Ouyang, H., 2016. Interactions of porcine circovirus 2 with its hosts. Virus genes, pp. 1-8.
Rossi, J. L. et al., 2000. Assembly of human papillomavirus type 16 pseudovirions in Saccharomyces cerevisiae. Human gene therapy, 11(8), pp. 1165-76.
Sainsbury, F., Thuenemann, E. C. & Lomonossoff, G. P., 2009. pEAQ: versatile expression vectors for easy and quick transient expression of heterologous proteins in plants. Plant biotechnology journal, 7(7), pp. 682-693.
Sylla, S. et al., 2014. Protective immunity conferred by porcine circovirus 2 ORF2-based DNA vaccine in mice. Microbiology and immunology, 58(7), pp. 398-408.

1. A method for producing a porcine circovirus (PCV) pseudovirion in a plant cell, the method comprising the steps of:
(i) introducing into the plant cell:
(a) an expression vector comprising a first nucleic acid encoding a PCV capsid polypeptide; and
(b) a replicating vector derived from a ssDNA virus comprising at least two origin of replication (Ori) sequences recognised by a viral replication regulatory protein, the replicating vector further comprising a second nucleic acid encoding a heterologous polypeptide, and
(c) a third nucleic acid encoding a viral replication regulatory protein, wherein replication of the replicating vector is initiated by the viral replication regulatory protein, and wherein the second nucleic acid is operably linked to a regulatory sequence which allows for the expression of the heterologous polypeptide in a mammalian cell;
(ii) expressing the PCV capsid polypeptide and the viral replication regulatory protein in the plant cell, and
(iii) replicating the replicating vector from the Ori sequence recognised by the viral replication regulatory protein in the plant cell, in order to produce a high copy number of a pseudogenome comprising the second nucleic acid,
wherein the expressed PCV capsid polypeptides assemble, together with a single-stranded copy of the pseudogenome and encapsidate the pseudogenome to produce a PCV pseudovirion.

2. The method of claim 1, wherein the first nucleic acid is operably linked to regulatory sequences that allow for expression of the PCV capsid polypeptide in the plant cell.

3. The method of claim 1, wherein the viral replication regulatory protein is expressed from at least one of the group selected from:
(i) a nucleic acid sequence contained on the replicating vector;
(ii) a nucleic acid sequence contained on the at least one expression vector;
(iii) a nucleic acid sequence contained on an independent vector, not being the vector of (i) or (ii) above; or
(iv) a nucleic acid sequence integrated into the genomic DNA of the plant cell;
wherein expression of the viral replication regulatory protein in the presence of the replicating vector results in replication of the replicating vector to produce a high copy number of the pseudogenome in the plant cell.

4. The method of claim 1, wherein the second nucleic acid encoding the heterologous polypeptide, comprises a gene selected from the group consisting of a reporter gene, a therapeutic gene, a gene encoding an antigenic polypeptide, a gene encoding a hormone, an antibody or an enzyme.

5. The method of claim 4, wherein the gene encoding a heterologous polypeptide is a reporter gene selected from a luciferase gene, a secreted alkaline phosphatase gene, a gene encoding a fluorescent protein or a horse radish peroxidase gene.

6. The method of claim 1, further comprising a step of recovering the PCV pseudovirion from the plant cell.

7. An assay for detecting the presence of a neutralising antibody to PCV in a subject, the assay including the steps of:
(i) combining a PCV pseudovirion produced according to the method of claim 1, with a biological sample from the subject to form a biological sample composition, wherein the heterologous polypeptide is a reporter polypeptide; and
(ii) combining a PCV pseudovirion produced according to the method of claim 1, with a control biological sample, wherein the control biological sample does not contain a PCV neutralising antibody, to form a control sample composition, wherein the heterologous polypeptide is a reporter polypeptide;
(iii) contacting and incubating a mammalian cell capable of being infected with PCV with the biological sample composition of (i) or the control sample composition of (ii); and
(iv) assaying the expression of the reporter polypeptide;
wherein decreased expression of the reporter polypeptide in the mammalian cells contacted with the biological sample composition, as compared to mammalian cells contacted with the control sample composition is indicative of the presence of a PCV neutralising antibody in the biological sample.

8. The assay of claim 7, wherein the reporter polypeptide is selected from a luciferase gene, a secreted alkaline phosphatase gene, a gene encoding a fluorescent protein or a horse radish peroxidase gene.

9. The assay of claim 7, wherein the subject is a pig.

10. A PCV pseudovirion produced according to the method of claim 1, the PCV pseudovirion comprising a capsid, wherein the capsid comprises the PCV capsid protein, wherein the capsid encapsidates the pseudogenome comprising the second nucleic acid encoding the heterologous polypeptide, wherein the second nucleic acid is operably linked to a regulatory sequence that allows for the expression of the heterologous polypeptide in a mammalian cell, wherein replication of the replicating vector is initiated from the Ori sequence recognised by the viral replication regulatory protein, and wherein the PCV pseudovirion is produced in and recovered from the plant cell.

11. The PCV pseudovirion of claim 10, wherein replication of the pseudogenome may be initiated, in a mammalian cell infected by the PCV pseudovirion in the presence of a viral replication regulatory protein, wherein the viral replication regulatory protein is a replication associated (Rep) protein.

12. The PCV pseudovirion of claim 11, wherein the viral replication regulatory protein is encoded by a nucleic acid sequence operably linked to a regulatory sequence that allows for the expression of the regulatory protein in the mammalian cell, wherein the viral replication regulatory protein may be expressed from any one of the group consisting of:
(i) a nucleic acid sequence contained on the pseudogenome;
(ii) a nucleic acid sequence contained on an independent vector; or
(iii) a nucleic acid sequence integrated into the genomic DNA of the mammalian cell,
wherein expression of the viral replication regulatory protein in the mammalian cell results in the replication of the pseudogenome.

13. The PCV pseudovirion of claim 10, wherein the gene encoding the heterologous polypeptide is selected from the group consisting of a reporter gene, a therapeutic gene, a gene encoding an antigenic polypeptide, a gene encoding a hormone, an antibody or an enzyme.

14. A pharmaceutical composition comprising a PCV pseudovirion produced by the method of claim 1 and a pharmaceutically acceptable carrier or adjuvant.

15. A pharmaceutical composition comprising a PCV pseudovirion of claim 10 and a pharmaceutically acceptable carrier or adjuvant.


SEQUENCE LISTING (7 sequences)

SEQ ID NO: 1 - DNA, 2641 bp, Artificial Sequence
  Construct (excluding the YFP gene and BeYDV rep/repA gene) provided on pUC57 vector cloned into PvuI sites
cgatcgctgcagtgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgcccggggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaccggtatatacatatactcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaatagcattcattttatgtttcaggttcagggggaggtgtgggaggttttttaaagcaagtaaaacctctacaaatgtggtacccgggataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtcgactgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttggtttaaacgaattcgagctcggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttcgactctagaggatccccttaaatcgatatatacatatacaattgctcgaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatgtttaaaccgatcgctgcag

SEQ ID NO: 2 - DNA, 22 bp, Artificial Sequence
  LIR-specific forward primer
acgatgtgatggtatttgcccg

SEQ ID NO: 3 - DNA, 20 bp, Artificial Sequence
  SIR-specific reverse primer
cgtgcctctcctcatacgag

SEQ ID NO: 4 - DNA, 28 bp, Artificial Sequence
  BeYDV rep/repA-specific forward primer
gaaatcgatatgccttctgctagtaaga

SEQ ID NO: 5 - DNA, 27 bp, Artificial Sequence
  BeYDV rep/repA-specific reverse primer
cttcaattgtcagtgactcgacgattc

SEQ ID NO: 6 - DNA, 20 bp, Artificial Sequence
  YFP-specific forward primer
cccgacaaccactacctgag

SEQ ID NO: 7 - DNA, 20 bp, Artificial Sequence
  YFP- specific reverse primer
gtccatgccgagagtgatcc